(19) United States
(12) Patent Application Publication
(10) Pub. No.: US 2022/0202360 A1
(43) Pub. Date: Jun. 30, 2022

(54) Sleep Sensing and Monitoring Device

(71) Applicant: Withings, Issy Les Moulineaux (FR)

(72) Inventors: Nicolas AIMON, Nantes (FR); Thomas BEDETTI, Issy Les Moulineaux (FR); Paul EDOUARD, Paris (FR); Pierre BARTET, Bievres (FR); Varoun SANATH KOUMAR RADJA, Issy Les Moulineaux (FR); Paul-Edouard JUAN, Paris (FR)

(73) Assignee: Withings, Issy Les Moulineaux (FR)

(21) Appl. No.: 17/607,725

(22) PCT Filed: Feb. 19, 2020
(86) PCT No.: PCT/EP2020/054345

§ 371 Date: Oct. 29, 2021

(30) Foreign Application Priority Data
Jul. 12, 2019 (EP) 19315068.7

(51) Int. Cl. A61B 5/00; G16H 40/67; A61B 7/00

(52) U.S. Cl. CPC A61B 5/4818; G16H 40/67; A61B 5/6892; A61B 2562/02; A61B 5/7264; A61B 5/0022; A61B 2562/0247; A61B 7/003

(57) ABSTRACT

The disclosure is directed to a sensing device, configured to be installed in a bedding, for monitoring a user's sleep, the device comprising:
a sensing part, for acquiring/determining a value representative of an a force or pressure and/or a value representative of a variation of a force or pressure,
a housing comprising at least a pressure transducer and an electronic processing unit,
a microphone connected to the electronic processing unit,
wherein the electronic processing unit is configured to process first and second electrical signals delivered respectively by the microphone and the pressure converter, wherein the electronic processing unit is either configured to deduce locally at least a breathing disturbance therefrom or configured to send data representative of the first and second electrical signals to a remote device.

### FIELD OF THE DISCLOSURE

The present disclosure relates to devices and systems for sensing and monitoring sleep of a user.

Monitoring sleep of an individual user provides useful information for such user. Notably when the user may undergo some sleep disorder or trouble or poor quality sleep, sometimes without knowing it, in-depth monitoring can reveal some light or severe sleep disorder, and can help improve the user's sleep quality.

### BACKGROUND OF THE DISCLOSURE

There has been proposed various detection devices designed to allow sleep monitoring, such detection devices are usually placed in the bedding. More specifically, this type of detection device can be installed above a mattress and under a mattress pad or cover; it can also be installed under a mattress topper or below the main mattress or under any other layer; the person to be monitored is intended to be lying down on top of the detection device.

Such detection devices are configured to capture the user's movements and allow determining by ballistography analysis, from pressure changes induced by the user's movements, the heart rate and respiratory rate of the individual, and also data reflecting amplitude of breathing.

EP2873368 illustrates one example of such a device.

However, it is not possible with such detection devices to reliably detect certain sleep problematic phases like sleep apnea and sleep hypopnea.

### SUMMARY OF THE DISCLOSURE

According to one aspect of the present invention, it is disclosed a sensing device, configured to be installed in a bedding, for monitoring a user's sleep, the device comprising:
a sensing part, for acquiring/determining a value representative of a force or pressure and/or a value representative of a variation of a force or pressure,
a housing comprising at least a force or pressure converter and an electronic processing unit,
a microphone connected to the electronic processing unit,
wherein the electronic processing unit is configured to process first and second electrical signals delivered respectively by the microphone and the force or pressure converter, wherein the electronic processing unit is either configured to deduce locally at least a breathing disturbance therefrom or configured to send data representative of the first and second electrical signals to a remote device.

Thanks to this arrangement, the combined analysis of sounds and movements (either large, medium or tiny movements) can be used to readily and accurately detect/identify a breathing disturbance.

Under the term “force or pressure”, it shall be equivalently understood an effort exerted by the user body. Similarly, under the term “variation of a force or pressure”, it shall be understood an effort difference.

Under the term “breathing disturbance”, it shall be understood a sleep apnea sequence or a hypopnea sequence.

In use, the effort is exerted by the user's body (UB) lying on the sensing part.

Under the term “pressure converter”, it shall be understood any device able to convert an effort (mechanical force) into an electrical value, e.g. a voltage, to be processed by a control unit. Such pressure converter may be involved whatever the type of sensing part, either pneumatic, or piezoelectric, or otherwise.

It shall be understood that the sensing part can be either piezoelectric (one or more piezoelectric elements) or can be pneumatic (i.e. an air bladder for sensing air pressure therein).

In particular, the inventors have found out that the microphone, even though it is arranged within the housing which is intended to be placed in the bedding, give nonetheless very useful outputs. Preferably, the housing is a unique housing.

It shall be noted that the apnea can be a central apnea phenomenon or an obstructive apnea phenomenon, or mixed. In the central apnea phenomenon, the nervous system does not command the breathing cycle. In the obstructive apnea phenomenon, the nervous system does command the breathing cycle, but the mouth and/or larynx of the user prevent air passage, or hinder severely such air passage.

According to the American Academy of Sleep Medicine (2017), see Kapur V K, Auckley D H, Chowdhuri S, Kuhlmann D C, Mehra R, Ramar K, Harrod C G, Clinical practice guideline for diagnostic testing for adult obstructive sleep apnea: an American Academy of Sleep Medicine clinical practice guideline. J Clin Sleep Med. 2017; 13(3):479-504, hypopnea is defined as a reduction of 30% or more of the respiratory signal during 10 s or more, coupled with a reduction of at least 3% of the oxygen saturation and/or micro-awakening events. Apnea is defined by a reduction of 90% or more of the respiratory signal during 10 s or more. under the term “hypopnea”, we understand periods of reduction of the breathing flux during a sleep, by contrast with periods of complete obstruction of the breathing flux that is rather qualified as “apnea”.

It shall be noted that the promoted sensing arrangement is a fortiori capable for snoring detection and normal breathing detection.

In various embodiments of the invention, one may possibly have recourse in addition to one and/or other of the following arrangements, taken alone or in combination.

According to one aspect, the microphone (1) is housed into the housing (6). Said otherwise the microphone is integrated into/within the housing. Thereby, the mechatronics integration is optimized and the solution is cost effective. Further, the microphone is protected from mechanical constraints.

According to one aspect, the microphone (1) can be arranged in any part of the sensing device, for example the microphone (1) can be arranged either next to a connection cable (7) or integrated into a connector to provide power supply.

According to one aspect, the electronic processing unit may be configured to calculate a apnea/hypopnea index (AHI) over a night. The apnea hypopnea index (AHI) is the mean frequency of apnea and hypopnea events over a night expressed in events per hour.

Advantageously, this index gives a complete picture of the user behavior and type of disorder regarding apnea/hypopnea. This index represents an average of breathing disturbance occurrences over a complete period of sleep. This index proves to be a relevant index to qualify the user behavior and type of disorder he/she may have.

According to one aspect, the housing may have a thickness which is less than 20 mm, preferably less than 15 mm. The device can therefore be installed in a bed/bedding without exhibiting a large protrusion. The device can be undetectable and discreet. The user can roll over the housing without notable hindrance.

According to one aspect, the sensing part is formed as a sensory band, having a thickness which is less than the thickness of the housing. The sensing surface, which is the surface of the sensory band, can be large enough to accommodate various user's positions. The sensory band can be undetectable and discreet within the bedding.

According to one aspect, there may be provided a microphone hole arranged in the housing, and the sensitive portion of the microphone is arranged opposite the microphone hole. Thereby, the microphone faces outwardly and can thus efficiently sense the external sounds without barrier effect from the housing. Even though the housing and microphone are covered by a cover lining made in fabric, it permits sounds to pass through with little attenuation (no substantive attenuation from the cover lining).

According to one aspect, the microphone is fixed on a flexible printed circuit (FPC) and the FPC is glued on an internal face of the housing, next to the microphone hole. This variant is a simple solution to arrange the microphone at the housing wall, independently of the position of the mother board (PCB).

According to one aspect, the electronic processing unit may be configured to determine pressure changes from the second electrical signals, for example by derivative processing, digital or analog. Starting from pressure changes, even with low voltage signals, we may use amplification stage so to work on easy to handle signals. For pneumatic solution, working on pressure changes renders the downstream process independent of absolute pressure in the bladder.

According to one aspect, the sensing part comprises one or more piezoelectric elements and the pressure converter converts piezoelectric voltage into a converted voltage that can be inputted in the electronic processing unit. This forms a simple all electronic solution, no pump is required.

According to an alternative solution, the sensing part is pneumatic, and the pressure converter is formed as a pressure transducer that converts pressure values into a converted voltage that can be inputted in the electronic processing unit. Thereby, only one sensor can reliably sense the movements, whatever the position of the user.

According to an aspect regarding the pneumatic configuration, the sensing part comprises a pneumatic chamber, which may otherwise be called an air bladder, and the housing comprises a pump and a motor, and preferably the housing further comprises a purge valve. The pneumatic chamber can be inflated to an optimal pressure for the sensing function. The pneumatic chamber can be deflated before rolling the device and stowing. The pump is driven by the motor, controlled by the electronic processing unit.

According to an aspect regarding the pneumatic configuration, the sensing device may further comprise one or more pipes for fluidly coupling together at least the pneumatic chamber, the pump and the pressure transducer. Advantageously the piping is enclosed and protected within the housing.

According to one aspect, the sensing device exhibits a rectangular overall shape (LX, LY) with LX comprised between 50 mm and 800 mm, and LY comprised between 10 mm and 400 mm. The sensitive area can be large enough to accommodate various user's positions. It does not exceed popular bedding horizontal sizes and therefore can be aesthetically integrated into a bed.

According to one aspect, the sensing device may further comprise a rechargeable electric battery. Thereby, in operation, the device does not need to have a wire connection. Recharge can be carried out through a wire or through a magnetic flux coupling.

According to one aspect, a data set regarding sleep can be transmitted by the sensing device to a remote entity by a wireless coupler, Bluetooth, Wifi or similar.

According to one aspect, the sensing device may further comprise a connection cable with one or more wire and a connector. Such connector may be a USB connector or any type of plug to provide electrical supply to the sensing device.

According to one aspect, in order to deduce locally a breathing disturbance, the electronic processing unit may be configured to process the said first and second electrical signals by obtaining a plurality of one-dimension time series channels from the said first and second electrical signals, and by performing an analysis of the time series channels together using a trained machine learning algorithm. Thus, calculations can be performed in situ by the sensing device and the results can be sent directly from the sensing device to a remote device.

According to one aspect, the electronic processing unit may be configured to process the said first and second electrical signals by obtaining a plurality of one-dimension time series channels from the said first and second electrical signals, sending data representative of the plurality of time series channels to a remote device, and the remote device may be configured to perform an analysis of the time series channels together using a trained machine learning algorithm.

The information related to breathing disturbance events is contained in the patterns formed by the different time series channels together representing several features useful in detecting breathing disturbances. It gives more accurate results to consider the different features in terms of time series channels, rather than a list of vectors characterizing a state at given timestamps.

According to one aspect, a breathing disturbance (e.g. of apnea or hypopnea events) index in a predetermined time window is obtained after analysis, i.e. the number of breathing disturbance event per unit time. For example, AHI in a predetermined time window can be obtained.

According to one aspect, the trained machine learning algorithm is a one-dimensional Convolutional Neural Network (CNN).

Standard machine learning algorithm have difficulties dealing with time series channel inputs, whereas CNN gives with very good results. The use of CNN thus makes it possible to deduce breathing disturbances directly from the electronic processing unit.

According to one aspect, each time series channel may be obtained from the first electrical signal and/or the second electrical signal, for example after being filtered with a predetermined set of filters.

According to one aspect, the first electrical signal and the second electrical signal are sampled by the electronic processing unit at a first frequency between 20 Hz and 1 kHz, which result in a first primary series of the first electrical signal and in a second primary series of the second electrical signal, otherwise called “raw” series

According to one aspect, the first and second primary series are processed to give the above mentioned time series channels having less data points per second.

More precisely, a digital filter can be applied to the first primary series by the electronic processing unit, either a low pass filter, and/or a band pass filter and/or a high pass filter. The data time density of the outputted time series channels is comprised between 0.5 Hz and 10 Hz (analogous to a second frequency). In a simple embodiment, it can be a subsampling with respect to the first frequency.

Similar digital filter can be applied to the second primary series by the electronic processing unit.

This ‘subsampling’ digital filter gives better performances in particular when using a CNN: the overall size of CNN algorithm can be less than 500 kbytes in memory, and the calculation speed is increased. Thus, calculations can be performed in situ in efficient manner by the sensing device and results sent directly to a remote device.

According to one aspect, the plurality of time series channels may comprise at least four time series channels obtained from the first signal, and at least two time series channels obtained from the second signal.

The plurality of time series channels may include for example at least some of the following:
a respiration amplitude channel obtained from the first signal,
a movements channel obtained from the first signal,
a heart rate channel obtained from the first signal,
a heart rate variability channel obtained from the first signal,
a snoring volume channel obtained from the second signal, and
a snorting channel obtained from the second signal. During a breathing disturbance event, for example during an apnea event:
for snorers, the end of an apnea event is often accompanied by several snoring cycles which can be detected using the snoring volume channel,
a choking or snorting sound can also sometimes be heard as the breathing resumes, which can be detected using the snorting channel,
the amplitude of the respiration is lowered during an apnea event, which can be detected using the respiration amplitude channel,
short movement bursts tend to happen at as the breathing resumes, which can be detected using the movements channel,
heart rate tends to decrease during apnea events and increase sharply as the breathing resumes, which can be detected using the heart rate channel,
due to a reduction of the respiratory sinus arrhythmia, heart rate variability tends to decrease during an apnea event, and this can be detected using the heart rate variability channel.

Thus, the use of a combination or all of the above described time series channels, or other channels related to the above described ones, provides excellent results in detecting breathing disturbances like sleep apnea or hypopnea events.

The disclosure is also directed at a system comprising a sensing device as described above and a smartphone/remote device comprising an application to display results and user's history.

The disclosure is also directed at a method configured to be carried out by a system comprising a sensing device as described hereinabove, the sensing device comprising at least a sensing part, a microphone, a housing comprising at least a force or pressure transducer and an electronic processing unit, the method comprising:
installing the sensing device in a bedding, for monitoring a user's sleep,
acquiring/determining, by the sensing part, a value representative of a force or pressure and/or a value representative of a variation of a force or pressure,
processing, by the electronic processing unit, a first and second electrical signals delivered respectively by the microphone and the pressure converter of the sensing device,
either deducing locally, by the electronic processing unit at least a breathing disturbance therefrom or sending data representative of the first and second electrical signals to a remote device.

### BRIEF DESCRIPTION OF THE DRAWINGS

Other features and advantages of the invention appear from the following detailed description of two of its embodiments, given by way of non-limiting example, and with reference to the accompanying drawings, in which:

FIG. 1 illustrates a diagrammatical perspective view of a sensing device according to a first embodiment,

FIG. 2 shows an elevation sectional view of the device of FIG. 1,

FIG. 3 shows a transverse sectional view of the device of FIG. 1,

FIG. 4 illustrates a block diagram of the device of FIG. 1,

FIG. 5 illustrates a partial diagrammatical exploded view of the device of FIG. 1,

FIG. 6 illustrates a diagrammatical perspective view of the housing,

FIG. 7 shows an elevation sectional view of the housing, with notably the microphone arrangement,

FIG. 8 illustrates a diagrammatical graph of apnea basic index over the time calculated by means of the sensing device of FIGS. 1 to 7,

FIG. 9 illustrates a diagrammatical top view of a sensing device according to a second embodiment,

FIG. 10 shows a side sectional view of the device of FIG. 9,

FIGS. 11 and 12 illustrate diagrammatically two variants of a transverse sectional view of the device of FIG. 9,

FIG. 13 illustrates two variants for the sensing device of FIG. 1, wherein the microphone is disposed in variant locations on the sensing device,

FIG. 14 illustrates steps of a method to deduce breathing disturbances from the signals acquired by the sensing device,

FIG. 15 illustrates an example of processing of the signals in order to obtain a local breathing disturbance index using a trained machine learning algorithm.

### DETAILED DESCRIPTION OF THE DISCLOSURE

In the figures, the same references denote identical or similar elements. It should be noted that, for clarity purposes, some element(s) may not be represented at scale.

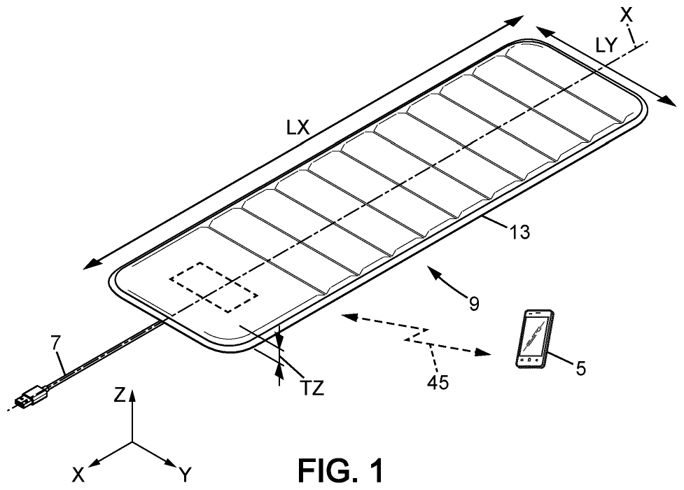

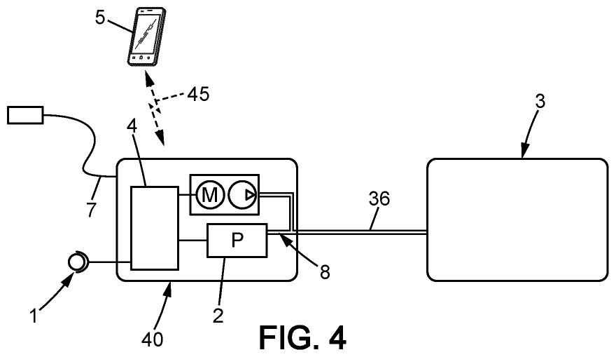

FIGS. 1 to 4 show a sensing device according to a first embodiment.

The sensing device 9 comprises a band 13 of fabric enclosing means for detecting apnea or hypopnea of a user during a sleep. The sensing device 9 may be stowable, either rolled or folded in three parts such that it is compact when not used. In the folded configuration, the sensing device 9 has a length of about 22 cm, a width of about 18 cm and a thickness of about 5 cm. In the rolled configuration, the sensing device 9 has a length of about 13 cm and a diameter of about 6 cm.

The sensing device 9 is distinct from the bedding mattress and distinct from the bedding frame. The sensing device 9 can be easily installed in the bedding and removed from the bedding.

The band 13 of fabric is intended to be installed under a mattress. When installed under the mattress, the user sleeping over the mattress would not be disturbed by the sensing device 9 presence. Indeed, the total thickness (TZ) of the band 13 is low enough to be undetectable under the mattress. For instance, the total thickness is about 20 mm or less. In use, it can be [10-20 mm]. In order to detect the apnea or hypopnea of the user during a sleep, the band 13 encloses a pneumatic chamber 3 acting as a sensing part.

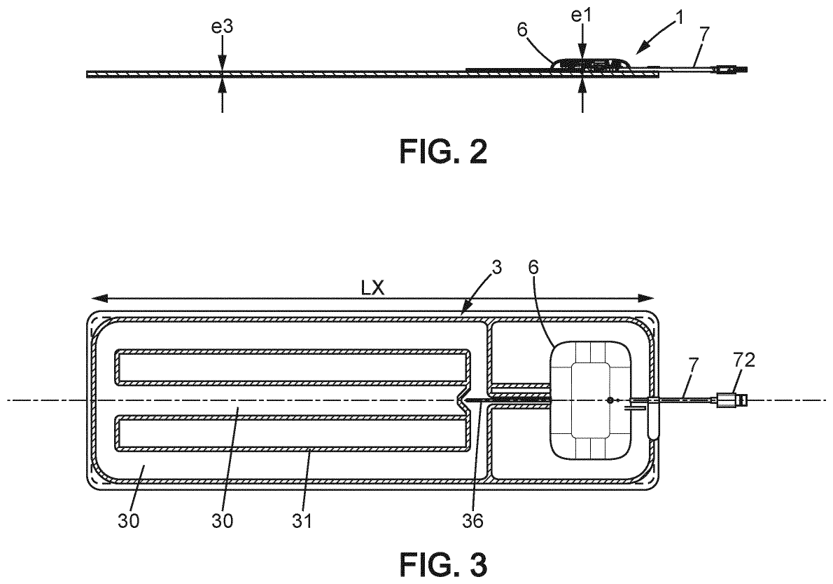

The pneumatic chamber 3 is represented more in detail on FIGS. 2 and 3. As one can see, the shape of the pneumatic chamber 3 is sensibly the same as the shape of the band 13, i.e. rectangular, and has an upper layer and a lower layer that vertically delimit the pneumatic chamber 3. The width LY of the rectangular shape is about 200 mm, and the length LX is about 600 mm. The pneumatic chamber 3 comprises some fluidly interconnected chambers portions 30 which are separated to each other by means of braces 31 joining two opposite faces of the chamber 3. For instance, the braces 31 are formed by heat melting the upper and lower layers of the pneumatic chamber 3. The chamber portions 30 therefore form some comfortable inflated tubes.

Some foam is included in the band 13 of fabric in order to make it further comfortable. Advantageously, the band 13 of fabric is removable and made into a material of fabric that is washable. For instance, the material is made of polyester and has a coating in thermoplastic polyurethane (TPU).

The chamber 3 has a total thickness e3 about 5 mm.

In order to get, analyze and transmit signals representative to apnea or hypopnea, the band 13 encloses a housing 6 comprising a pressure transducer 2. The pressure transducer 2 is connected with the chamber 3 via an air tube 8. The air tube 36 enables air connection even if the chamber 3 is pressed near the housing 6. Hence, the pressure can be transmitted through the air tube 36 whatever the user position lying on the bedding.

Alternatively, the air variation can pass through a channel coupling the air chamber with the pressure transducer, said channel having an anti-collapse rod therein.

The housing 6 further comprises electronic means. The electronic means namely comprise an electronic processing unit 4. The electronic means are further adapted for communicating with a smartphone 5, as represented by the dotted-line arrow 45. The electronic means are also connected to a connection cable 7 comprising a wire and a USB connector 72 in order to be able to electrically feed the sensing device 9.

In this configuration, only the free end of connection cable 7 protrudes outside the bedding. In a battery supplied configuration, the sensing device 9 can be fully integrated and hidden inside the bedding (beneath or above the mattress).

Further, the housing 6 comprises a microphone 1, also referred to as an audio sensor. The microphone is configured to acquire sounds, and in particular breathing or snoring sounds.

Both the microphone 1 and the pressure transducer 2 are electronically connected to the electronic processing unit 4.

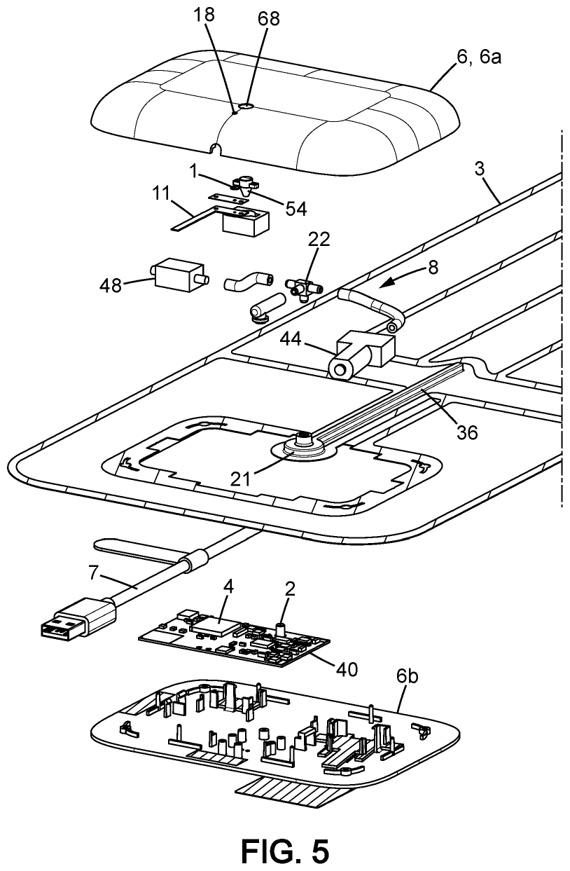

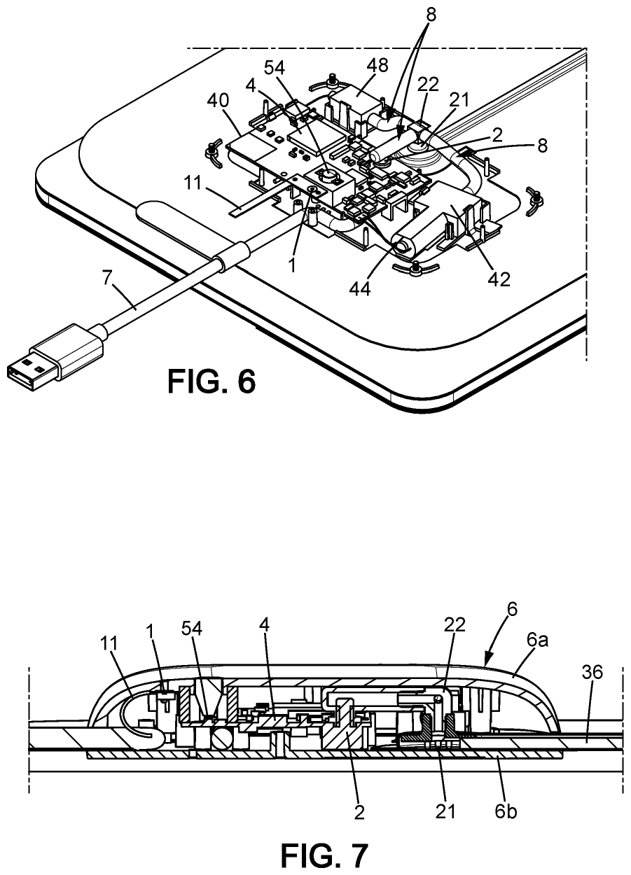

FIGS. 5 to 7 represent more in detail the housing 6. The housing 6 is made of plastic. The thickness e1 of the housing 6 is less than 15 mm.

The housing 6 comprises an upper shell 6a and a lower shell 6b that cooperate together in order to form an unfoldable rigid protection for the electronic means. For instance, the upper shell 6a and lower shell 6b are assembled by snap-fit assembly method or by ultrasonic weld assembly.

Inside the rigid protection, the housing 6 also comprises a Printed Circuit Board (PCB) 40. The pressure transducer 2 and the electronic processing unit 4 are disposed and connected to the PCB 40. The microphone is disposed higher than the plan surface of the PCB 40, on a support near the internal surface of the upper shell 6a. The microphone 1 is connected to the electronic processing unit 4 by means of a foldable flexible circuit board (FPC) 11. The upper shell 6a comprises a hole 18 formed on the supper surface in order to face the microphone 1 and enable the microphone 1 to get the sounds from the outside of the housing 6.

Therefore, the housing 6 forms a complete and secured enclosure that houses the microphone 1, the pressure converter 2 and the electronic processing unit 4.

Advantageously, a light emitting device (LED) 54 can also be included in the housing 6, and disposed on the PCB 40, in order to produce a lighting signal when the sensing device 9 is in use. Advantageously, the upper shell 6a further comprises a lighting hole 68 facing the LED 54 in order to diffuse the light through the plastic of the housing 6.

The upper shell 6a may further comprise a reset hole for accessing a switch electronically connected to the PCB 40.

One can see on FIG. 6 that the chamber 3 is connected to an input of a pneumatic connector 21 through the air tube 36. The pneumatic connector 21 has a “T” shape three-channel output 22, each of the channels being fluidly connected to a pipe 8.

One of the pipes 8 is fluidly connected to a purge valve 48, such that the chamber 3 forms an air bladder. One of the pipes 8 is fluidly connected to a pump 42 able to inflate the chamber 3. A motor 44 is provided for rotating the pump 42. One of the pipes 8 is fluidly connected to the pressure transducer 2 in order to convert detected pressure into voltage.

All the pipes 8 and fluidly connected pneumatic connector 21, purge valve 48, pump 42 and pressure transducer 2 are also enclosed and protected within the same unique housing 6.

The chamber 3 may be inflated prior to be used during the sleep time, for instance prior the night, by means of the pump 42 and the motor 44.

Therefore, when lying on the mattress, the weight and movements of the user act on the pneumatic, which results on pressure variations. The microphone gets the sounds of the surroundings, namely breathes or snoring, and converts them into a first voltage. The pressure transducer 2 simultaneously gets the pressure variations and converts it into a second voltage.

The electronic processing unit 4 is configured for analyzing both the first and second voltage and to calculate an apnea/hypopnea index (AHI) over a night, for the user.

Said otherwise, the electronic processing unit 4 is configured to process together a first signal delivered from the pressure transducer 2 and a second signal delivered from the microphone 1. In one embodiment, the first signal and second signals are processed locally within the sensing device 9 by the electronic processing unit 4. Alternatively, the electronic processing unit 4 is configured for sending, after at least some conditioning, the first and second voltage to the smartphone 5 which is configured either to calculate the AHI or to send the first and second voltage to a remote server which is configured to perform said calculations.

For instance, the calculations are performed by means of a trained artificial intelligence (AI). The AI is trained by feeding an algorithm with data sets comprising tagged records of sounds and of pressure variations. The tags enable to classify the records. For instance, a set comprising a record of sounds and a record of pressure variation of a user during a whole night may be associated to the following tags:
weight of the user,
age of the user,
type of apnea or AHI measured by a polysomnograph and read by a trained medical professional.

More specifically, for the AI training purpose, records of sounds and pressure variation are performed by a sensing device according to the invention, on a plurality of study patients. A doctor identifies, for each study patient, a type of breathing disturbance thanks to medical monitoring of various physiological signals, called polysomnography (PSG). The most importantly signals of the PSG used for the diagnosis of sleep apnea are the air flux in the breathing of the study patient, snoring, movements of the thorax and abdomen, and the O2 saturation in the blood of the study patient. The different types of breathing disturbances identified by the doctor may be hypopnea, apnea of mix of hypopnea and apnea, and may be numbered over a night by the AHI. The different types of apnea may be obstructive, central, or mixed. This gives a reference database, which can be improved/increased over time, and patient monitoring.

Therefore the doctor may tag the records of sounds and of pressure variation in association with the profile, i.e. for instance the age, gender and weight, of each study patient.

In use, the trained AI is able to estimate, or “predict”, an AHI from the pressure and sound recordings of the device of the present invention.

The user may install a dedicated application on his/her smartphone 5. The dedicated application may display graphs of variation of the frequency of apnea/hypopnea episodes in the course of the night. The application may also display the AHI, which represents the average number of apnea/hypopnea episodes per hour in the night.

The application may also display a graph of an apnea basic index 12 over the time on the screen of the smartphone 5.

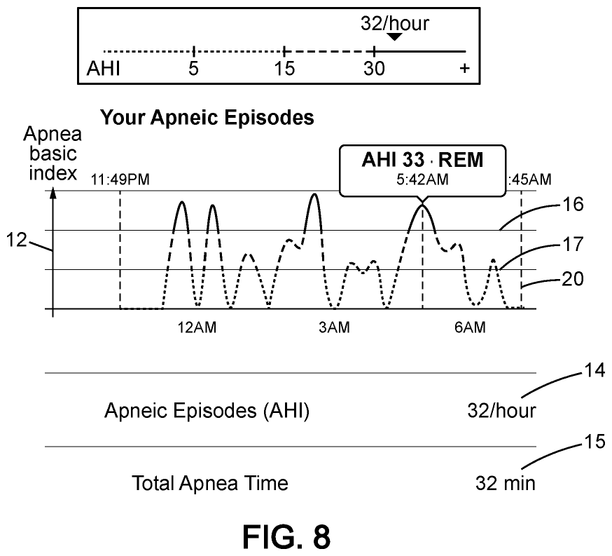

The graphical user interface of the application is pictured on FIG. 8.

The apnea basic index 12 represents the number 14 of likely apneic episodes during an elementary time division. As represented, for a time division equal to an hour, the number 14 in the displayed example is 32 per hour.

The graph of apnea basic index 12 is divided in three categories of number 14 of episodes per hour: low number 20, medium number 17 and high number 16, corresponding to the medical classification of gravity of the apnea, respectively, light apnea (0 to 15 episodes), moderate apnea (15 to 30 episodes) and severe apnea (over 30 episodes). A “total apnea time” represents a cumulated time lapse 15 over the sleep during which the number 14 is high, i.e. is in the high part 16 on the graph. When the total apnea time is high, for instance is over 30 min, then the probability that the user suffers from apnea—in other words, that the user could be diagnosed as an apneic person—is high.

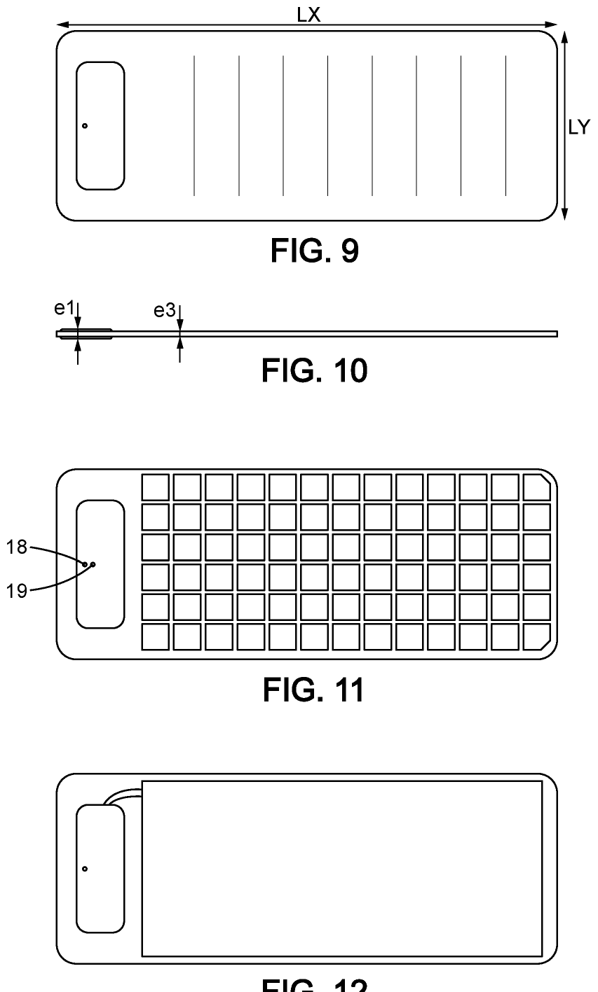

The FIGS. 9 and 10 show an alternative embodiment to the embodiment described with reference to the FIGS. 1-4. The overall difference is that the sensing part is not made from a pneumatic chamber 3 but made from a series of parallel lined piezoelectric elements. One can see on the figures that the parallel lines are transversally extending. In another embodiment (not represented on the figures), one can design parallel lines longitudinally extending as well.

The pressure converter 2 converts the piezoelectric voltage into a converted voltage that can be inputted in the electronic processing unit 4.

The FIG. 11 shows an alternative embodiment to the embodiment described with reference to the FIGS. 1-4. The only difference is that the sensing part is not made from a pneumatic chamber 3 but made from a matrix of piezoelectric elements. The pressure converter 2 in this embodiment converts the piezoelectric voltage into a converted voltage that can be inputted in the electronic processing unit 4.

The FIG. 12 shows a further alternative embodiment. The only difference with the embodiment described with reference to the FIG. 11 is that the sensing part is made from a unique large piezoelectric element.

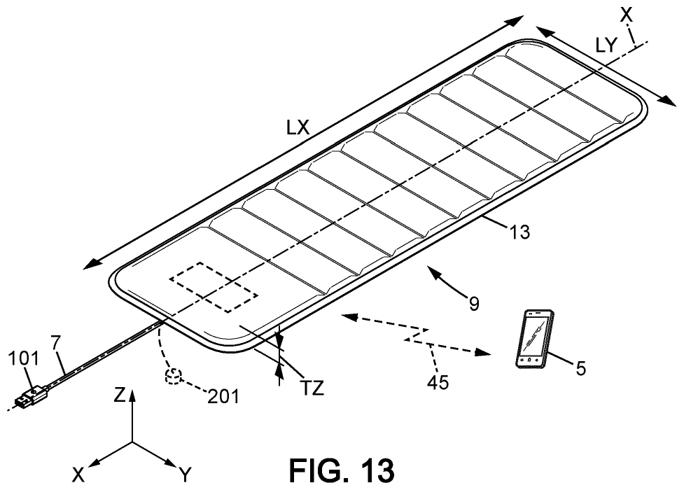

One can see on the FIG. 13 two alternative dispositions of the microphone 1, by comparison with the above description.

For instance, instead of being enclosed in the housing 6, a microphone 201 may be either disposed on the connection cable 7.

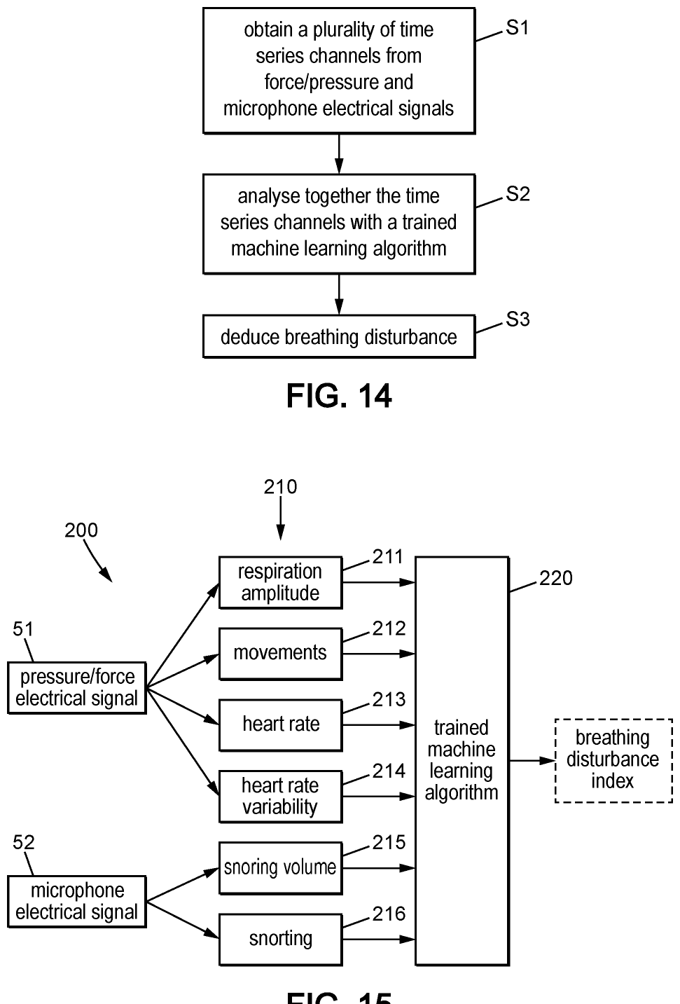

According to another arrangement, there is provided a microphone 101 disposed on the USB connector 72. The microphone 101 may have the same functional behaviors as per above embodiments. An example method (computer-implemented method) to deduce breathing disturbances with the sensing device 9 is illustrated at FIG. 14. The processing 200 of the signals in order to obtain a local breathing disturbance index is further described at FIG. 15.

At step S1, a plurality of time series channels 210 from force or pressure electrical signal 51 (first signal) and microphone electrical signal 52 (second signal) are obtained. Step S1 may be performed directly by the electronic processing unit 4 or by a remote device after the electronic processing unit 4 has sent data representative of the first 51 and second 52 electrical signals.

The first electrical signal and the second electrical signal are sampled by the electronic processing unit 4 at a first frequency between 20 Hz and 1 kHz, which result in a first primary series of the first electrical signal denoted 51 and in a second primary series of the second electrical signal denoted 52. First and second primary series are otherwise called “raw” series.

Then, the first and second primary series are processed to give the above mentioned time series channels 210 having less data points per second (smaller size in memory) than the raw series.

More precisely, a digital filter can be applied to the first primary series by the electronic processing unit 4, either a low pass filter, and/or a band pass filter and/or a high pass filter. The data time density of the outputted time series channels 210 is comprised between 0.5 Hz and 10 Hz (analogous to a second frequency, lower than the first frequency). It is not excluded to use a simple subsampling to obtain the above mentioned time series channels 210. However various types of digital filters can be used to reveal and/or extract, from the raw series, particular features representative of the bio signals and bio cycles of the user.

The plurality of time series channels 210 includes in this example: a respiration amplitude channel 211 obtained from the first signal 51, a movements channel 212 obtained from the first signal 51, a heart rate channel 213 obtained from the first signal 51, a heart rate variability channel 214 obtained from the first signal 51, a snoring volume channel 215 obtained from the second signal 52, and a snorting channel 216 obtained from the second signal 52. This set of time series channels 210 permits accurate detection of breathing disturbance events.

At step S2, the time series channels 210 are analyzed together using a trained machine learning algorithm 220 (or trained AI), i.e. they are inputted together in the trained machine algorithm 220. Step S2 may be performed directly by the electronic processing unit 4 or by a remote device after the electronic processing unit 4 has sent data representative of the first 51 and second 52 electrical signals.

In an example, the trained machine learning algorithm 220 is a one-dimensional convolutional neural network, which is convenient to deduce breathing disturbances from time series channels 210.

At step S3, breathing disturbance is deduced from the analysis done at step S2, either locally by the electronic processing unit 4 or by a remote device. When using a convolutional neural network and time series channels, the output of the algorithm may be a number of breathing disturbance events (e.g. apnea/hypopnea events) per unit time (or in other words: a breathing disturbance index) in a predetermined time window, which can then be used to calculate AHI in an elementary time window or over a night.

1. A sensing device, configured to be installed in a bedding, for monitoring a user's sleep, the device comprising:
a sensing part, for acquiring/determining a value representative of a force or pressure and/or a value representative of a variation of a force or pressure,
a housing comprising at least a force or pressure converter and an electronic processing unit,
a microphone connected to the electronic processing unit,
wherein the electronic processing unit is configured to process first and second electrical signals delivered respectively by the microphone and the force or pressure converter, wherein the electronic processing unit is either configured to deduce locally at least a breathing disturbance therefrom or configured to send data representative of the first and second electrical signals to a remote device.

2. The sensing device according to claim 1, wherein the microphone is housed into the housing.

3. The sensing device according to claim 1, wherein the electronic processing unit is configured to calculate a apnea/hypopnea index over a night.

4. The sensing device according to claim 1, wherein the housing has a thickness which is less than 20 mm.

5. The sensing device according to claim 1, wherein the housing has a thickness which is less than 20 mm, wherein the sensing part is formed as a sensory band, having a thickness which is less than the thickness of the housing.

6. The sensing device according to claim 1, wherein the microphone is housed into the housing, wherein there is provided a microphone hole arranged in the housing, and the sensitive portion of the microphone is arranged opposite the microphone hole.

7. The sensing device according to claim 1, wherein the sensing part comprises one or more piezoelectric elements and the pressure converter converts piezoelectric voltage into a converted voltage that can be inputted in the electronic processing unit.

8. The sensing device according to claim 1, wherein the sensing part is pneumatic, and the pressure converter is formed as a pressure transducer that converts pressure values into a converted voltage that can be inputted in the electronic processing unit.

9. The sensing device according to claim 1, wherein the sensing part is pneumatic, and the pressure converter is formed as a pressure transducer that converts pressure values into a converted voltage that can be inputted in the electronic processing unit, wherein the sensing part comprises a pneumatic chamber and the housing comprises a pump and a motor.

10. The sensing device according to claim 1, wherein said device exhibits a rectangular overall shape (LX, LY) with LX comprised between 50 mm and 800 mm, and LY comprised between 10 mm and 400 mm.

11. The sensing device according to claim 1, wherein, in order to deduce locally a breathing disturbance, the electronic processing unit is configured to process the said first and second electrical signals by obtaining a plurality of one-dimension time series channels from the said first and second electrical signals, and by performing an analysis of the time series channels together using a trained machine learning algorithm.

12. The sensing device according to claim 1, wherein, in order to deduce locally a breathing disturbance, the electronic processing unit is configured to process the said first and second electrical signals by obtaining a plurality of one-dimension time series channels from the said first and second electrical signals, and by performing an analysis of the time series channels together using a trained machine learning algorithm, wherein the trained machine learning algorithm is a one-dimensional convolutional neural network.

13. The sensing device according to claim 11, wherein, in order to deduce locally a breathing disturbance, the electronic processing unit is configured to process the said first and second electrical signals by obtaining a plurality of one-dimension time series channels from the said first and second electrical signals, and by performing an analysis of the time series channels together using a trained machine learning algorithm, wherein a breathing disturbance index in a predetermined time window is obtained after said analysis of the time series channels.

14. A system comprising a sensing device according to claim 1, and a smartphone/remote device comprising an application to display results and user's history.

15. A method configured to be carried out by a system comprising a sensing device according to claim 1, the sensing device comprising at least a sensing part, a microphone, a housing comprising at least a force or pressure transducer and an electronic processing unit, the method comprising:
installing the sensing device in a bedding, for monitoring a user's sleep,
acquiring/determining, by the sensing part, a value representative of a force or pressure and/or a value representative of a variation of a force or pressure,
processing, by the electronic processing unit, a first and second electrical signals delivered respectively by the microphone and the pressure converter of the sensing device,
either deducing locally, by the electronic processing unit at least a breathing disturbance therefrom or sending data representative of the first and second electrical signals to a remote device.

16. A method according to claim 15, further comprising, by the electronic processing unit, calculating a apnea/hypopnea index over a night.

17. A method according to claim 15, further comprising, by the electronic processing unit, in order to deduce locally a breathing disturbance;
processing the said first and second electrical signals, by obtaining a plurality of one-dimension time series channels from the said first and second electrical signals, and
performing an analysis of the time series channels together using a trained machine learning algorithm.

18. A method according to claim 15, further comprising, by the electronic processing unit, in order to deduce locally a breathing disturbance;
processing the said first and second electrical signals, by obtaining a plurality of one-dimension time series channels from the said first and second electrical signals, and
performing an analysis of the time series channels together using a trained machine learning algorithm, wherein the trained machine learning algorithm is a one-dimensional convolutional neural network.

19. A method according to claim 15, further comprising, by the electronic processing unit, in order to deduce locally a breathing disturbance;
processing the said first and second electrical signals, by obtaining a plurality of one-dimension time series channels from the said first and second electrical signals, and
performing an analysis of the time series channels together using a trained machine learning algorithm,
wherein the plurality of time series channels may comprise at least four time series channels obtained from the first signal, and at least two time series channels obtained from the second signal.

20. A method according to claim 15, further comprising, by the electronic processing unit, in order to deduce locally a breathing disturbance;
processing the said first and second electrical signals, by obtaining a plurality of one-dimension time series channels from the said first and second electrical signals, and
performing an analysis of the time series channels together using a trained machine learning algorithm,
wherein the plurality of time series channels may comprise at least four time series channels obtained from the first signal, and at least two time series channels obtained from the second signal, wherein the plurality of time series channels include at least some of the following in the list consisting in:
a respiration amplitude channel obtained from the first signal,
a movements channel obtained from the first signal,
a heart rate channel obtained from the first signal,
a heart rate variability channel obtained from the first signal,
a snoring volume channel obtained from the second signal, and
a snorting channel obtained from the second signal during a breathing disturbance event, for example during an apnea event:
for snorers, the end of an apnea event is often accompanied by several snoring cycles which can be detected using the snoring volume channel,
a choking or snorting sound can also sometimes be heard as the breathing resumes, which can be detected using the snorting channel,
the amplitude of the respiration is lowered during an apnea event, which can be detected using the respiration amplitude channel,
short movement bursts tend to happen at as the breathing resumes, which can be detected using the movements channel,
heart rate tends to decrease during apnea events and increase sharply as the breathing resumes, which can be detected using the heart rate channel,
due to a reduction of the respiratory sinus arrhythmia, heart rate variability tends to decrease during an apnea event, and this can be detected using the heart rate variability channel.

21. A method according to claim 15, further comprising, by the electronic processing unit, in order to deduce locally a breathing disturbance;
processing the said first and second electrical signals, by obtaining a plurality of one-dimension time series channels from the said first and second electrical signals, and
performing an analysis of the time series channels together using a trained machine learning algorithm, wherein a breathing disturbance index in a predetermined time window is obtained after said analysis of the time series channels.

22. A method according to claim 15, further comprising, by the electronic processing unit, in order to deduce locally a breathing disturbance;
processing the said first and second electrical signals, by obtaining a plurality of one-dimension time series channels from the said first and second electrical signals, and
performing an analysis of the time series channels together using a trained machine learning algorithm, wherein a breathing disturbance index in a predetermined time window is obtained after said analysis of the time series channels,
calculating a apnea and/or hypopnea
